(19) United States
(12) Patent Application Publication
(10) Pub. No.: US 2019/0088353 A1
(43) Pub. Date: Mar. 21, 2019

(54) DYNAMIC MEDICATION AND PROTOCOL MANAGER FOR HOME

(71) Applicant: KONINKLIJKE PHILIPS N.V., Eindhoven (NL)

(72) Inventors: John Francis HUMPHRYS, Needham, MA (US); Kim CHAPMAN, Salem, NH (US)

(21) Appl. No.: 15/534,019

(22) PCT Filed: Nov. 18, 2015
(86) PCT No.: PCT/IB2015/058914

§ 371 Date: Jun. 8, 2017

Related U.S. Application Data
(60) Provisional application No. 62/090,659, filed on Dec. 11, 2014.

(51) Int. Cl. G16H 20/10; G16H 10/60; G06F 3/0481

(52) U.S. Cl. CPC G16H 20/10; G06F 3/04817; G16H 10/60

(57) ABSTRACT

A mobile device (7) executes an application program (7a) to perform a chronic disease management support method including the operations of: maintaining a disease management schedule of self-care tasks including at least a medication schedule; displaying a time interval (420) of the disease management schedule including icons (422) representing self-care tasks to be performed during the time interval; generating an alert when the time for a self-care task arrives; recording at the mobile device whether each self-care task is performed on schedule; and in response to user selection of an icon representing a self-care task via a user interface of the mobile device, displaying information pertaining to the self-care task represented by the icon. The display may include an analog clock face (420) representing a twelve-hour time interval which indicates a current time, with superimposed icons (422) representing self-care tasks to be performed during the twelve-hour time interval.

### BACKGROUND

The following relates generally to systems and methods for assisting a patient at home in adhering to his or her treatment program.

A common goal of medical care for a chronic condition that is (or may be) incurable, or is likely to recur, is to enable a person to live a normal life at home in spite of the condition (or in spite of a high likelihood of recurrence). To this end, even patients with relatively serious chronic medical conditions, such as diabetes, chronic obstructive pulmonary disease (COPD), chronic heart failure (CHF), bipolar disorder, clinical depression, pain management, or the like, are usually either never admitted to a hospital as an in-patient, or are released from the hospital after a short stay during which their condition is stabilized. When the patient returns home, he or she is given tasks to perform to provide self-care for the chronic condition. Such tasks may include (using diabetes as an illustrative chronic condition): administering insulin shots on a strict schedule; taking other medications orally; monitoring diet and adjusting insulin intake accordingly; monitoring blood sugar levels using a glucometer and adjusting insulin intake accordingly; performing other physiological monitoring and tracking general physical condition (e.g. recognizing dizziness as a possible symptom of blood sugar imbalance); and so forth.

Performing these various self-care tasks in accord with a strict schedule is essential to successfully manage the chronic condition. However, the patient is busy living his or her life, which distracts from paying close attention to the various self-care tasks. Some tasks can also be complex and/or tedious: in the diabetic case, complex tasks include operating the glucose meter and then recording the reading on a regular basis, estimating carbohydrate intake (especially in settings such as restaurants in which the carbohydrate content of food items may be unavailable), keeping tracking of insulin injection locations, and so forth. Furthermore, the chronic condition itself may be an impediment to performing self-care tasks. For example, if a diabetic fails to take an insulin shot on schedule, a resulting low blood sugar condition may impair the patient's memory, or leave the patient in a confused or even unconscious state.

Ideally, the patient engaged in self-care in a home setting is not isolated, but rather has a support network including parents, a spouse or other family, and medical professionals such as the patient's physician or pharmacist. In practice, however, these individuals are unable to monitor the patient's self-care on a constant basis yet even a single missed insulin dose or other delayed self-care task can have serious consequences. Indeed, these interactions commonly rely upon the out-patient taking the initiative to reach out to a support person. For example, the patient usually must contact the pharmacist to refill a prescription if the patient forgets or neglects to do so, the medication may run out leading to missed doses.

Current scheduling systems for out-patient chronic disease management typically provide task lists organized in formats such as Gantt charts (a type of bar chart illustrating a project schedule), Program Evaluation Review Technique (PERT) charts, tabular lists, etc. However, the output is voluminous and does not consider the dynamic nature of the healthcare environment. Patients need tests, medications, and procedures each according to the patient's schedule. Although there may be some flexibility in completing some tasks, tasks are constantly being added, changed, or removed in response to health conditions. These scheduling systems also typically do not provide a mechanism by which the patient's support network is leveraged to assist in performing various self-care tasks.

Additionally, there are known systems of organizing medications such as plastic pill box organizers with boxes labeled for each day of the week. However, such systems can be are cumbersome, do not automatically update, and usually address only one facet of the patient's self-care (e.g., pill box organizers assist in organizing oral medication intake, but cannot assist in other aspects such as insulin injection, dietary monitoring, and blood sugar monitoring.

The following provides a new and improved systems and methods for managing out-patient self-care.

### SUMMARY

In some illustrative embodiments, a chronic disease management support system is disclosed. A mobile device comprising a microprocessor executes an application program to perform a chronic disease management support method including the operations of: maintaining a disease management schedule of self-care tasks including at least a medication schedule; displaying a time interval of the disease management schedule including icons representing self-care tasks to be performed during the time interval; generating an alert when the time for a self-care task arrives; recording at the mobile device whether each self-care task is performed on schedule; and in response to user selection of an icon representing a self-care task via a user interface of the mobile device, displaying information pertaining to the self-care task represented by the icon. In a disclosed embodiment, the displaying comprises displaying an analog clock face representing a twelve-hour time interval which indicates a current time, and superimposing icons representing self-care tasks to be performed during the twelve-hour time interval on the analog clock face at locations on the analog clock face corresponding to the scheduled times for the self-care tasks.

In a non-transitory storage medium embodiment, the storage medium stores instructions readable and executable by a mobile device that includes a microprocessor to perform a method including: receiving, at the mobile device, prescription information of a patient including a medication type and a medication dosage; storing the prescription information in a memory of the mobile device; and based on the stored prescription information, displaying a scheduled time for a dose of medication on a display of the mobile device.

In a method embodiment, a chronic disease management support method comprises: displaying a twelve-hour time interval of a disease management schedule of self-care tasks on a mobile device by displaying an analog clock face representing the twelve-hour time interval which analog clock face displays a current time, and superimposing icons representing self-care tasks to be performed during the twelve-hour time interval on the analog clock face at locations on the analog clock face corresponding to the scheduled times for the self-care tasks; generating an alert when the time for a self-care task arrives; recording at the mobile device whether each self-care task is performed on schedule; and in response to user selection of an icon representing a self-care task via a user interface of the mobile device, displaying information pertaining to the self-care task represented by the icon. The method may further comprise: replicating the display of the twelve hour time interval on a monitoring mobile device including the display of the analog clock face and the superimposed icons; communicating the record of whether each self-care task is performed on schedule from the mobile device to the monitoring mobile device; and displaying at the monitoring mobile device at least a portion of the record of whether each self-care task is performed on schedule.

One advantage resides in reducing the likelihood of medication errors, missed self-care tasks, or the like.

Another advantage resides in providing a holistic tool for managing interrelated self-care tasks.

Another advantage resides in facilitating trusted third party monitoring and intervention in out-patient self-care.

Another advantage resides in simplified recordation of information, such as meal information, and linking such recordation with performing associated tasks.

Still further advantages of the present disclosure will be appreciated to those of ordinary skill in the art upon reading and understanding the following detailed description. It is to be understood that a given embodiment may achieve none, one, two, more, or all of these advantages

The invention may take form in various components and arrangements of components, and in various steps and arrangements of steps. The drawings are only for purposes of illustrating the preferred embodiments and are not to be construed as limiting the invention.

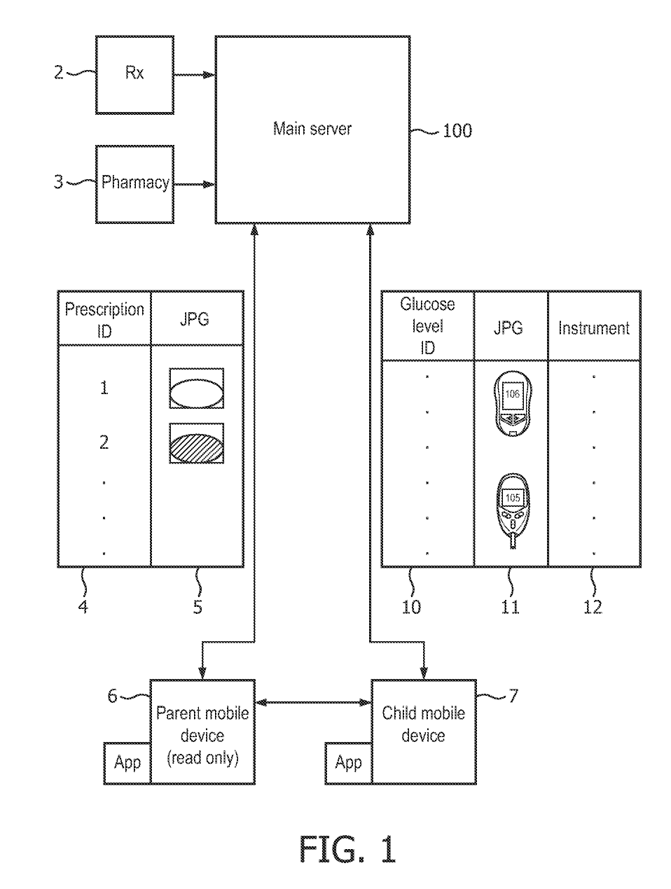

FIG. 1 diagrammatically shows an embodiment of a medical scheduling system.

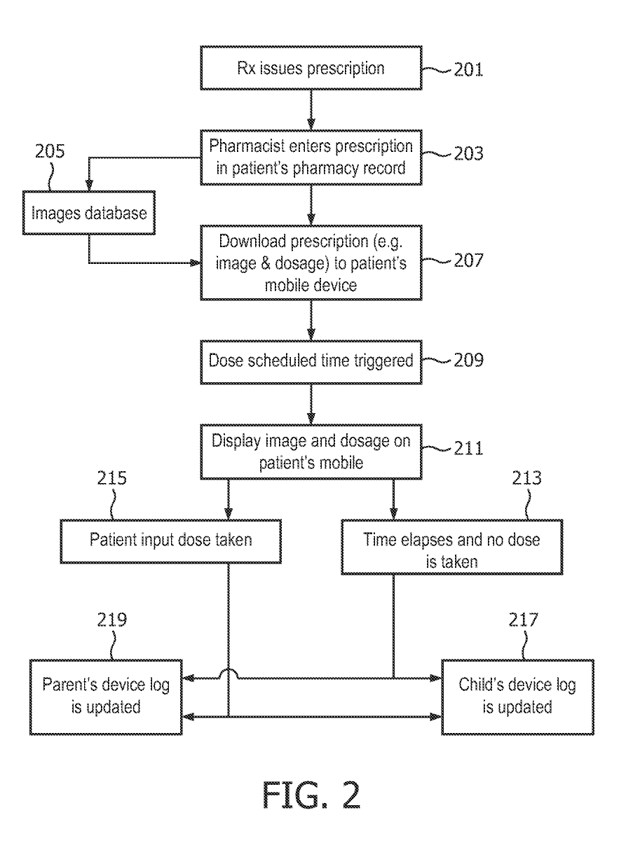

FIG. 2 flowcharts an embodiment.

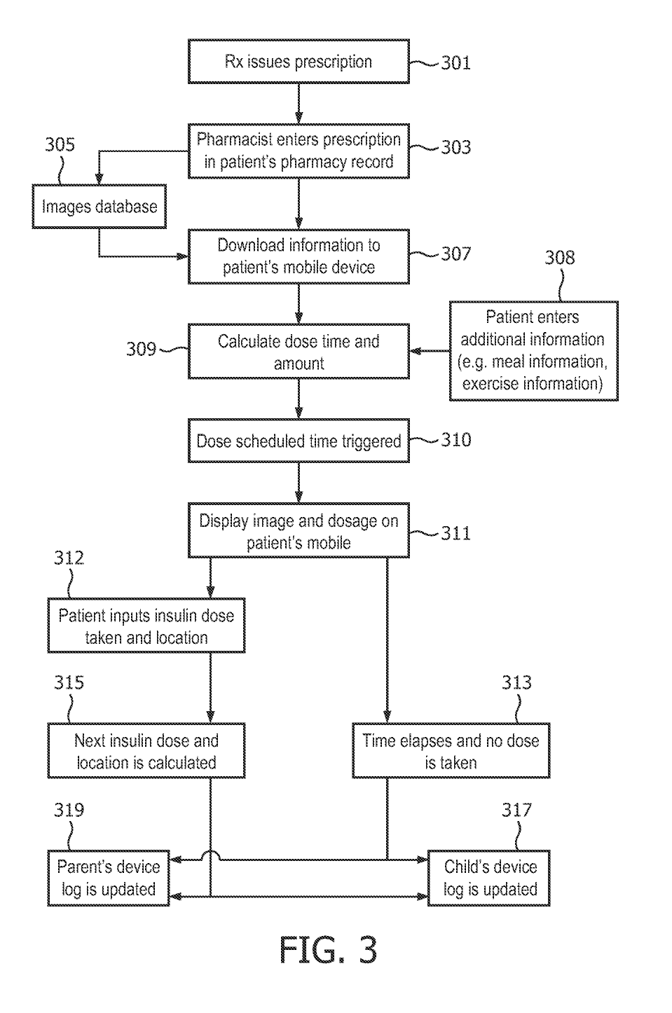

FIG. 3 flowcharts another embodiment.

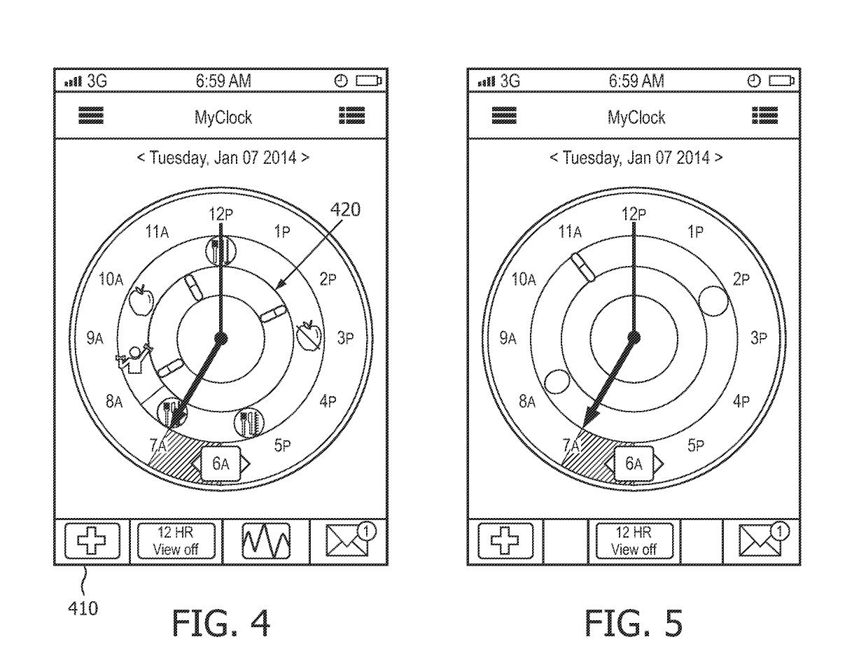

FIG. 4 shows an embodiment including an analog clock face.

FIG. 5 shows another embodiment including an analog clock face.

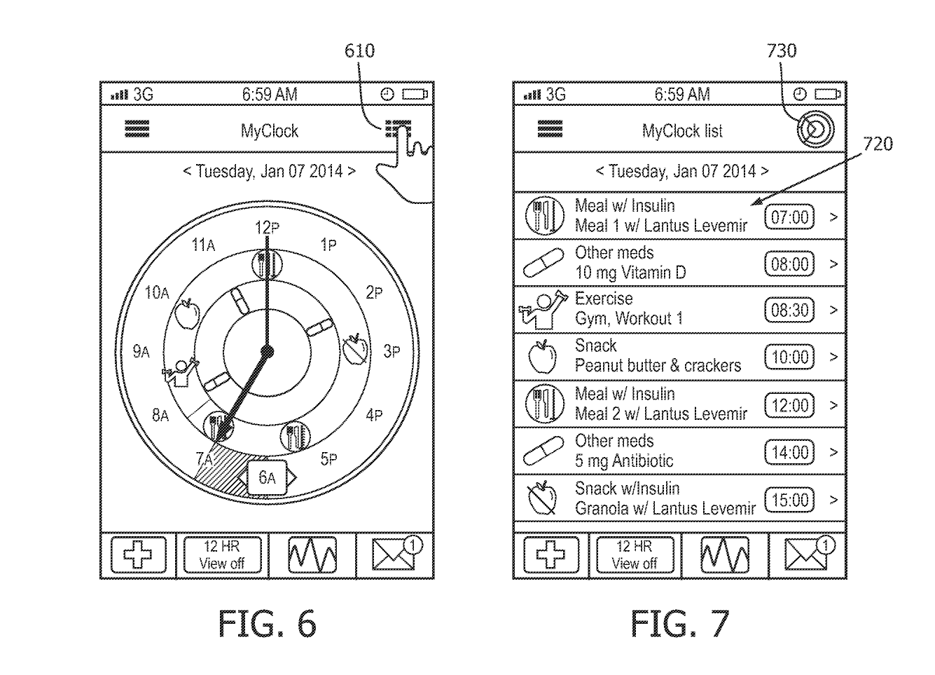

FIG. 6 shows another embodiment including an analog clock face.

FIG. 7 shows an embodiment including a list view.

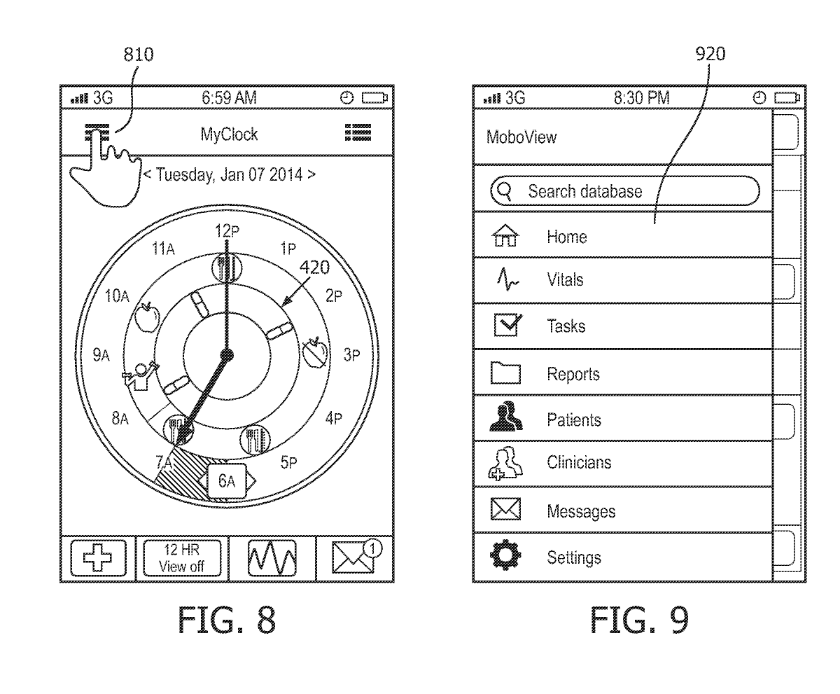

FIG. 8 shows another embodiment including an analog clock face.

FIG. 9 shows an embodiment of a main menu of choices.

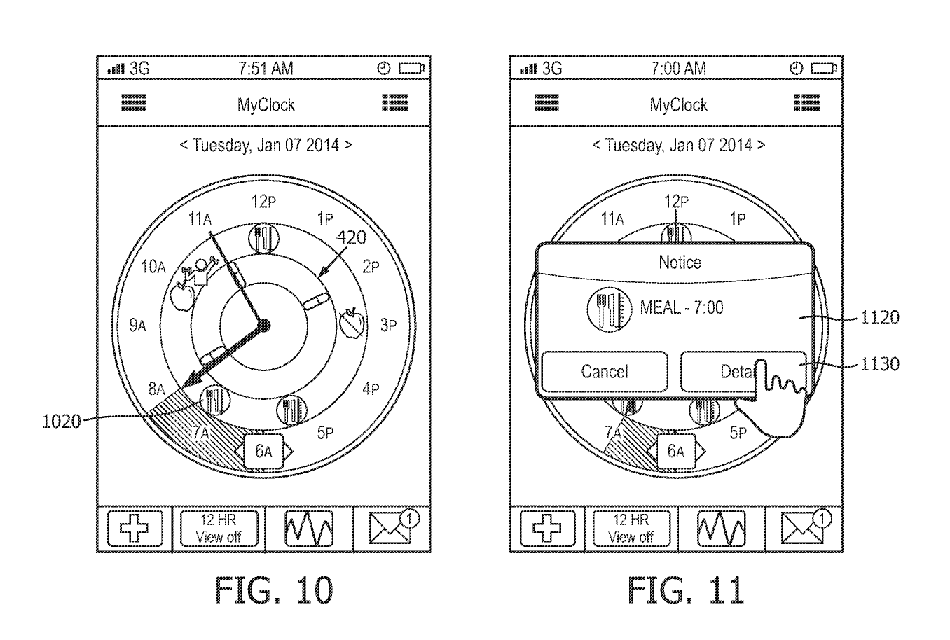

FIG. 10 shows another embodiment including an analog clock face.

FIG. 11 shows an embodiment of a notification.

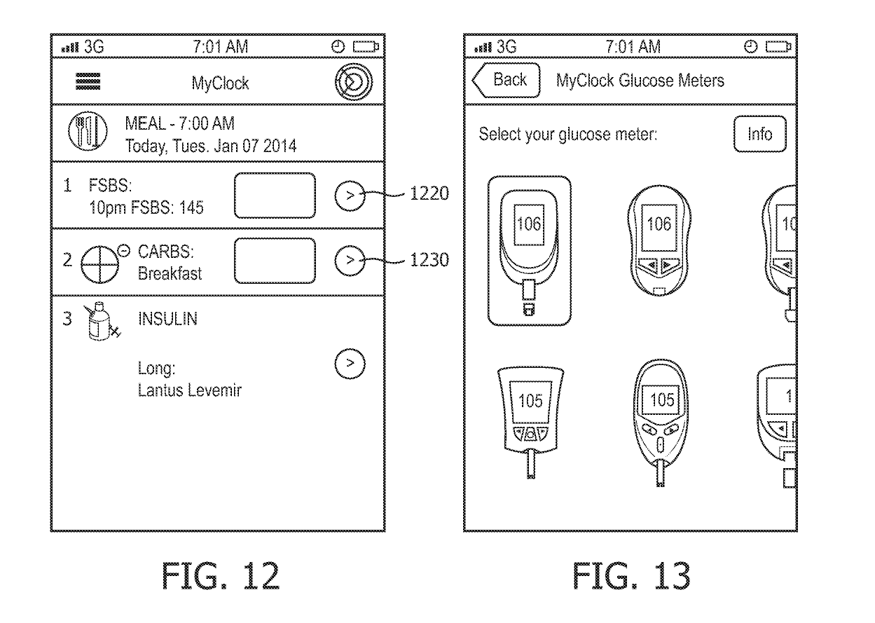

FIG. 12 shows an embodiment of details that may appear when a details button is pressed.

FIG. 13 shows an embodiment allowing a user to select a glucose meter.

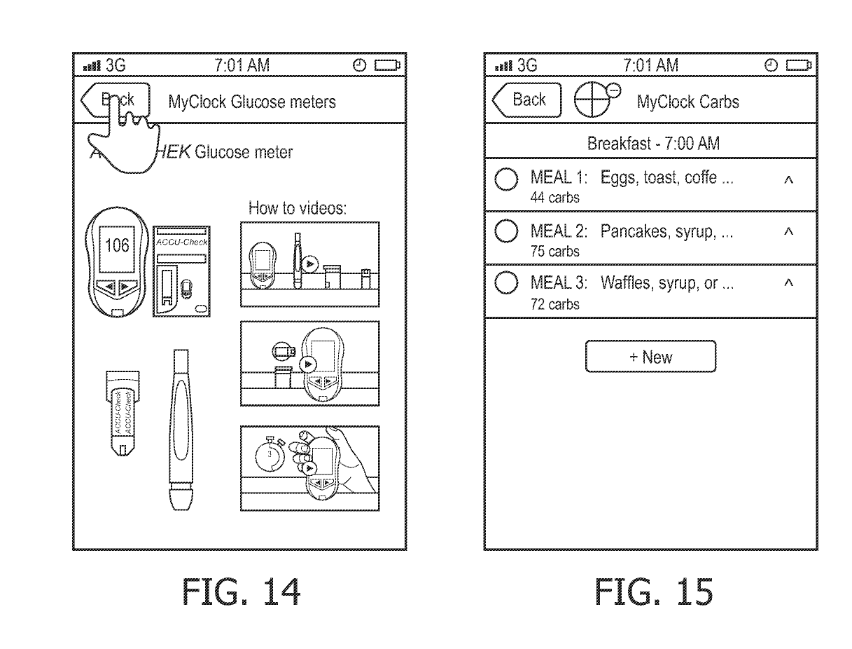

FIG. 14 shows an embodiment of a screen that may be displayed when a glucose meter is selected.

FIG. 15 shows an embodiment of a screen allowing a user to select a meal.

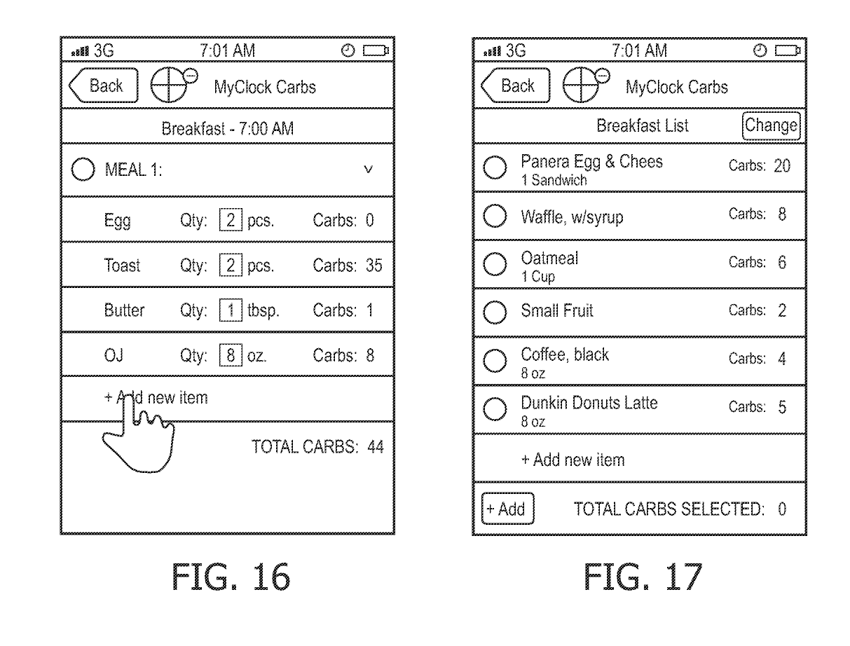

FIG. 16 shows an embodiment of a user entering meal information.

FIG. 17 shows an embodiment of a screen allowing a user to enter meal information.

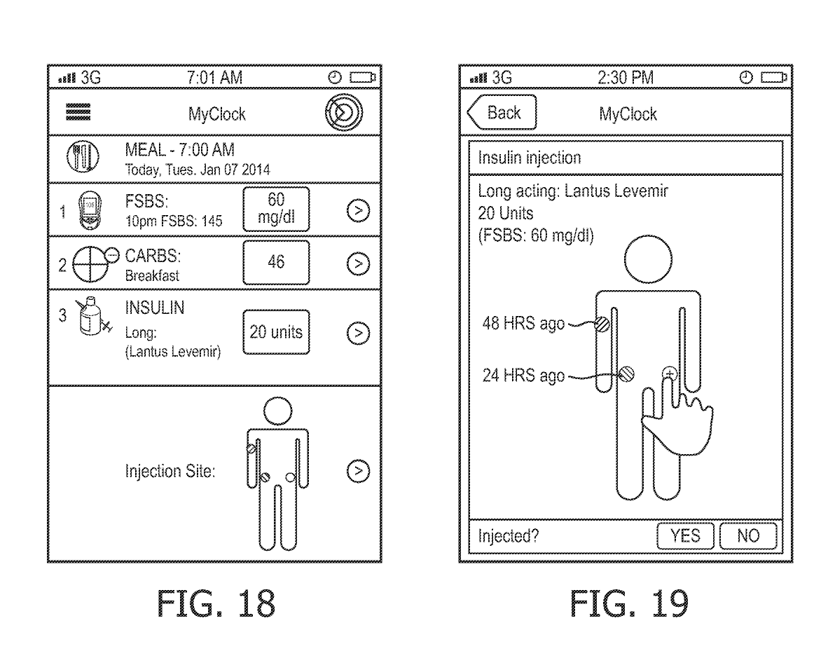

FIG. 18 shows an embodiment of a screen with instructions for a user to take an amount of insulin.

FIG. 19 shows an embodiment of a screen allowing a user to enter insulin injection information.

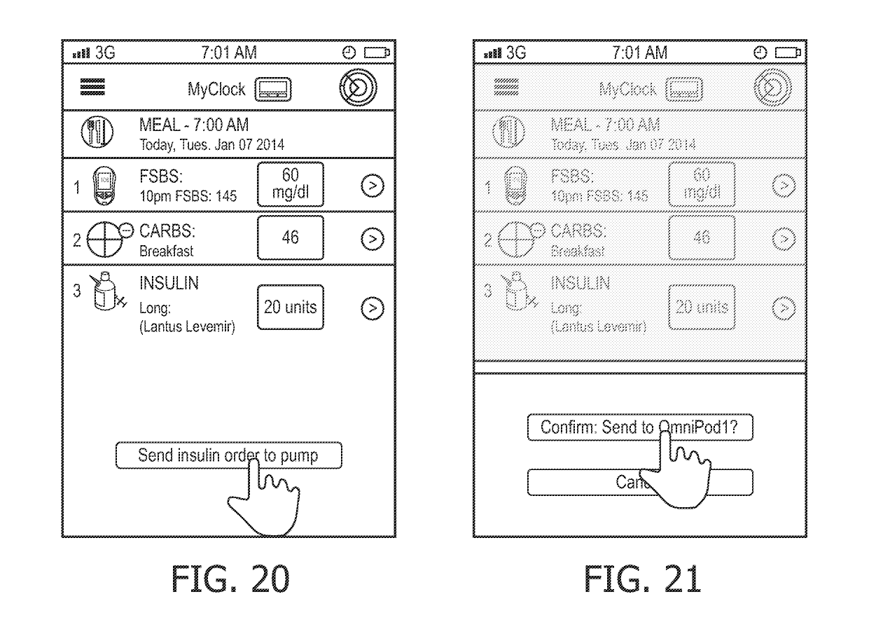

FIG. 20 shows an embodiment of a screen relating to automatic insulin injection.

FIG. 21 shows another embodiment of a screen relating to automatic insulin injection.

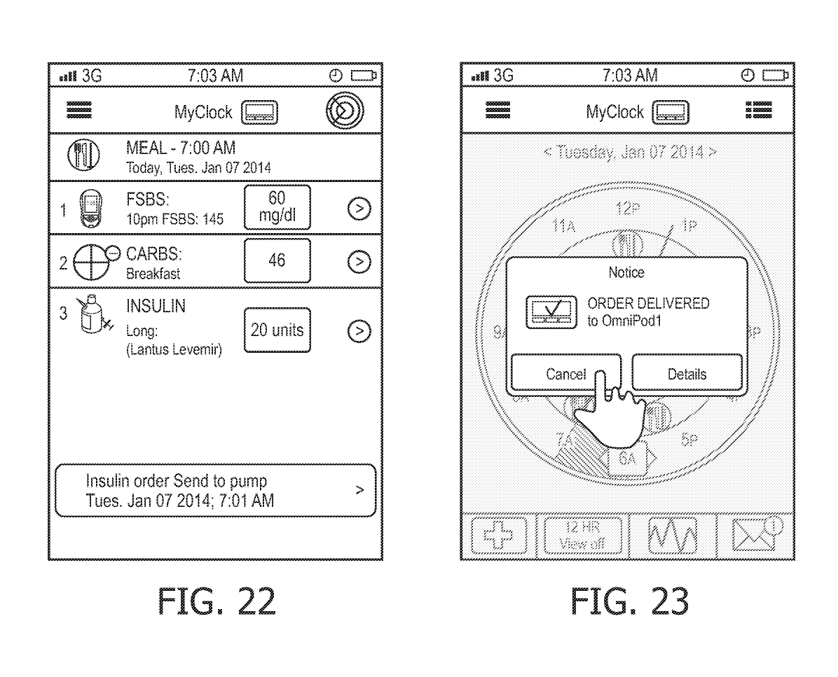

FIG. 22 shows another embodiment of a screen relating to automatic insulin injection.

FIG. 23 shows another embodiment of a screen relating to automatic insulin injection.

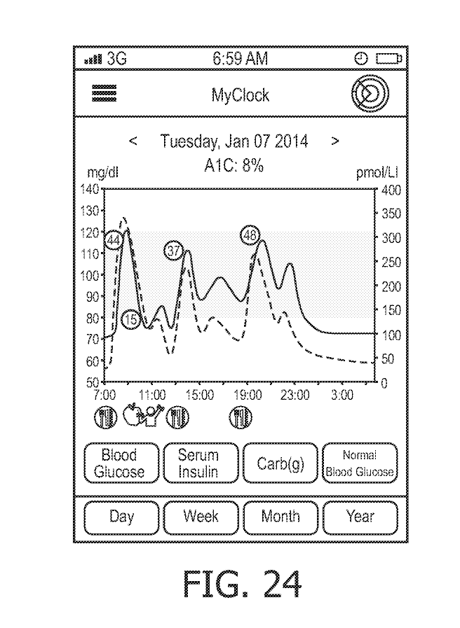

FIG. 24 shows an embodiment of a graphical display of a blood glucose amount and other information.

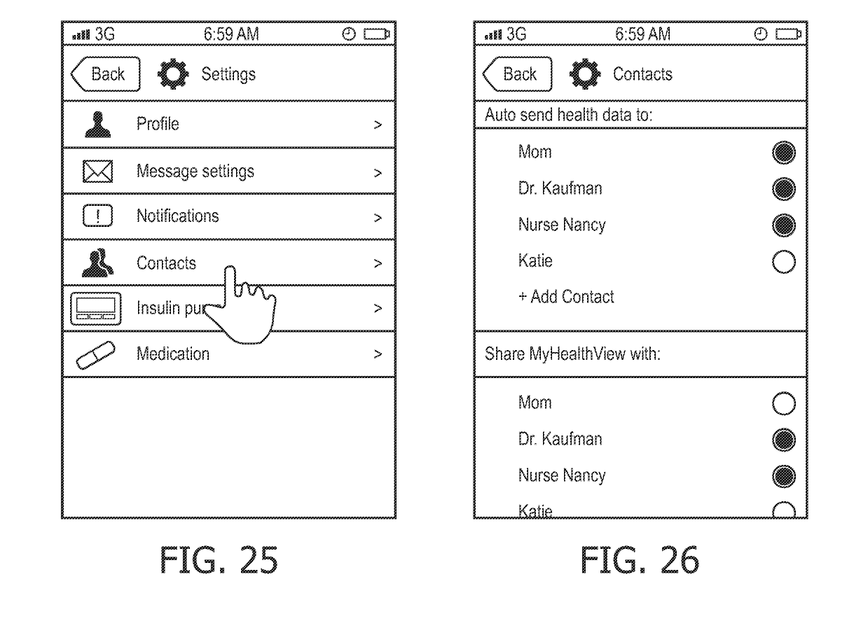

FIG. 25 shows an embodiment of a screen relating to a user entering contact information.

FIG. 26 shows an embodiment of a screen related to contact information.

### DETAILED DESCRIPTION

An objective of the techniques described herein is to help patients better manage their chronic medical condition in the home, including reducing medication errors and other self-care task performance errors. The devices described herein provide a convenient way to view all tasks included in a medical protocol prescribed by their doctor and/or a medication schedule with one or more medications as well as providing means for viewing medication dosages, adverse reactions, previous injection sites, and administration techniques. This is especially valuable following a discharge from the hospital for patients who frequently are confronted by new and complex medication or protocol schedules. Therefore, advantageously, before a patient is discharged from a stay at a hospital, the patient may be provided the tool as described herein.

These techniques are particularly useful in treatments that include, for example, administering medications on a strict schedule, monitoring blood sugar levels, and coordinating meals, snacks, and exercise with the prescribed medications. Furthermore, they leverage the patient's support network in carrying out the self-care by providing the ability to notify trusted personal contacts regarding whether self-care tasks such as testing and medication administration have been performed. Still further, tasks are coordinated with specific devices such as insulin pumps. The patient's self-care support tool is also coordinated with medical personnel, such as the pharmacist and/or physician, to leverage their expertise in supporting the self-care.

With reference to FIG. 1, a main server 100 receives prescription information from a medical professional 2 (e.g., a doctor or nurse) or pharmacy 3. The prescription information is sent to mobile device 7 running a child application program (“app”) 7a. In the illustrative example the patient is a child with diabetes, hence the patient mobile device 7 is a child mobile device. More generally, the patient mobile device 7 may be utilized by an adult or pediatric patient having a chronic condition such as diabetes, chronic obstructive pulmonary disease (COPD), chronic heart failure (CHF), bipolar disorder, clinical depression, cystic fibrosis, attention deficit hyperactivity disorder (ADHD), or so forth. Further shown in the illustrative example depicted in FIG. 1 is a parent mobile device 6 running a read-only app 6a. More generally, the patient may be supported by a trusted third party, typically a parent, spouse, or other relative although the trusted third party may alternatively be an unrelated trusted third party such as a friend of the patient or a school nurse. The prescription information may include prescription ID information 4 and/or prescription image information 5. Main server 100 may also send other information relevant to the patient's self-care, such as glucose level ID information 10, glucose meter image information 11, and instrument information 12.

The mobile devices 6, 7 can, by way of illustration, be cellular telephones (i.e. cell phones), tablet computers, personal data assistant (PDA) mobile devices, or so forth that includes a microprocessor and ancillary electronic hardware (e.g. RAM). The devices preferably have wireless communication capability via a cellular telephone network, a WiFi network, or the like. However, it is contemplated for the app 7a to remain operable in an “airplane” mode or other unconnected mode, albeit without those features requiring wireless communication. The apps 6a, 7a may, by way of illustrative example, be apps running under the iOS mobile operating system (available from Apple Corp., Cupertino, Calif., USA), the Android mobile operating system (available from Google Inc., Mountain View, Calif., USA), or the like. In a suitable installation procedure, the app 6a, 7a may be downloaded from the app store of the relevant device/operating system, and when first opened the app is registered with (or logged into) the main server 100. The app 6a running on the parent (or other trusted third party) mobile device 6 is different from the app 7a running on the child (or other patient) mobile device 7 in that the app 6a operates in a “read only” mode to monitor app 7a via a wireless communication link 14. While drawn in diagrammatic FIG. 1 as directly connecting the mobile device 7 to mobile device 6, in practice this wireless communication connection may be indirect, for example via the main server 100 (e.g. data may be transferred from mobile device 7 to the server 100 where it is relayed to mobile device 6) or via an intermediate cellular relay tower or the like.

It is also to be understood that the provision of a trusted third party with the mobile device 6 running the read-only app version 6a is optional, and may be omitted if no third party monitoring is to be performed. Conversely, it is also contemplated to have two or more trusted third parties with respective instances of the mobile device 6 running read-only app 6a in communication with the patient mobile device 7. If trusted third party monitoring is provided via one or more mobile devices 6, suitable patient privacy mechanisms are preferably put into place. For example, access to the patient device 7 by the monitoring device 6 may be permitted only upon some affirmative “agreement” operation performed by the patient using the patient device 7, and all apps 6a, 7a are suitably prevented from unauthorized access by password protection, fingerprint identification security of the respective devices 6, 7, or the like. (Such privacy concerns may be less applicable in the illustrative case in which the trusted third party is a parent of a child/patient for example, HIPAA regulations generally allow a parent to have access to the medical records about his or her child but at least password protection or the like is preferably employed with the apps 6a, 7a). Moreover, while the trusted third party device 6 is described as having a “read-only” relationship to the monitored patient device 7, some communication in the direction from the device 6 to the device 7 is also contemplated. For example, the parent may be able to send messages to the child's device via the app 6a. Additionally or alternatively, if the devices 6a, 7a are cell phones or the like, then the parent device 6 may be used in its conventional manner to telephone the device 7 or to send a text message from the mobile device 6 to the mobile device 7.

The trusted third party monitoring, if provided, yields various benefits. It permits the third party to identify when the patient has failed to perform a self-care task so that the trusted third party can contact the patient to alleviate the error. Further, the trusted third party can verify correctness of data entered by, or actions taken by, the patient. For example, the parent via the device app 6a can see the insulin dose calculated by the child 7a to verify that it is correct (or at least that it is within a reasonable range of values). If the trusted third party is the patient's doctor, then more detailed analyses are contemplated, and the doctor may, for example, decide to telephone the patient to discuss the chronic disease management if the doctor believes, based on data observed by the doctor's instance of the read-only app 6a, that some change to the disease management protocol should be made.

With continuing reference to FIG. 1 and with further reference to FIG. 2, a process of updating medication information in the child's mobile device 7 is described. In step 201, the doctor 2 issues a prescription for a medication. In step 203, the pharmacy 3 enters the prescription in a patient's pharmacy record. Optionally, in step 205, the prescription information may be sent to an images database where it is correlated with an image of the prescribed medication, for example including an image of the actual pill (possibly cut in two if in accord with the prescribed dose). In step 207, the prescription information is received by the mobile device 7 (and more particularly by the app 7a) either directly from the pharmacy or from the images database, and is stored in a solid state memory, flash memory, secure digital (SD) card, or other electronic memory of the mobile device 7. In step 209, a scheduled time for the patient to take a medication is triggered. In step 211, an image of a medication is displayed at the scheduled time, optionally along with instructions for self-administering the medication. If the patient inputs information indicating that medication has been taken (e.g., step 215), then a child's device log of the app 7a is updated at step 217 and a parent's device log of the app 6a is also updated at step 219. On the other hand, if time elapses and no dose is taken (e.g., step 213) the child's device log is updated at step 217 and the parent's device log is updated at step 219.

FIG. 3 flowcharts a process of updating insulin administration information in the child's mobile device 7 and copying that information to the parent's mobile device 6. In one context, at the beginning of this process the physician (or other medical professional) and the patient set up the app together. They would set a schedule on the app that is recommended for that particular patient. Thereafter, in step 301, the child's doctor 2 issues an insulin prescription for the diabetic child. In step 303, the pharmacy 3 enters the insulin prescription in the pharmacy record. Optionally, in step 305, the insulin prescription information may be sent to an images database where it is correlated with an image of a prescribed insulin medication. In step 307, the insulin prescription information may be received by the child's mobile device 7 either directly from the pharmacy 3 or from the images database. At step 308, the child may enter additional information. For the illustrative insulin treatment directed to diabetes, it is especially useful if the patient enters information that may be used in a calculation of a required insulin amount (e.g., meal and exercise information). In step 309, a dose time and amount are calculated by the app 7a. In step 310, an insulin dose scheduled time is triggered. In step 311, an image of the insulin medication is displayed. In step 312, the child inputs the insulin dose actually administered via injection, along with the injection location; and a next insulin dose and location is calculated at step 315. Alternatively, if the patient does not input information, time will elapse (e.g., step 313). Based on the preceding events, the child's device log for app 7a will be updated at step 317 and the parent's device log for app 6a is updated at step 319.

FIG. 4 shows a typical display presented by the app 7a on the child's mobile device 7, which is constructed around an analog clock face 420, for a diabetic patient that both manages their diabetes as well as maintains a schedule with other medications. A cross button 410 can be selected (e.g. via a touch screen of the mobile device 7) to provide instructions for emergency situations related to a specific disease such as diabetes. Additionally, various icons 422 are superimposed on the analog clock face 420. The icons 422 indicate when various self-care events or other events related to the diabetes management are scheduled to occur these events may, for example, include scheduled meals, scheduled exercise, insulin injection times, insulin/snack compound events, and oral medication administration times.

FIG. 5 shows a display similar to that of FIG. 4, but relating to a treatment plan for a chronic condition in which the treatment involves only orally administered medications.

FIG. 6 shows a representation of a user of the mobile device 7 (e.g. the diabetic child) tapping list icon 610. If the user taps the list icon 610, a list view 720 is displayed as shown in FIG. 7, which lists each event icon from the clock face view of FIGS. 4 and 6 along with a description of the event and its time of scheduled occurrence. This allows the user to view and scroll through all the items shown on the clock face of FIG. 6. From the list view 720, the user may return to the clock face by tapping the clock-face icon 730 in the top, right hand corner of the display in FIG. 7. It will be appreciated that the clock view of FIGS. 4 and 6, on the one hand, and the list view of FIG. 7 on the other hand, provide the diabetic child with both an overview and a convenient representation of the time frame for next occurrence of certain events (via the clock face view) and more detailed explanation of each event (via the list view).

FIG. 8 shows a representation of a user tapping menu icon 810. If the user taps the menu icon, a main menu of choices 920 is displayed as shown in FIG. 9.

Some further operations that can be performed using the illustrative user interface of the app 7a are next described.

In one aspect, picture information (e.g., jpeg) is received (e.g. from a pharmacy, a database, a doctor's office or so forth) of the prescription bottle and/or a picture of the actual pill, capsule, drops, inhaler, cream, etc. The picture information illustrates the specific dosage prescribed to the patient to reduce dosage errors. For example, if the prescription called for 10 mg and the prescribed tablet was only available in 20 mg dosages, then a picture of the tablet could be shown for identification along with a picture of the tablet cut in half which is the correct dose for the patient.

FIG. 10 shows the display of FIGS. 4 and 6 for the diabetic child, where the current time has progressed past 7:00 am. As seen in FIG. 10, this means the current time has passed the 7:00 am scheduled time for breakfast and insulin, as indicated by the breakfast meal/insulin icon 1020. The breakfast meal/insulin icon has turned grey, indicating that the user has recorded into the app that they have had their breakfast and completed their insulin injection.

FIG. 11 illustrates a notification 1120 in the form of a pop-up window that automatically appears when it is time to perform a task on the clock (here, eating the breakfast meal indicated by the icon 1020 of FIG. 10). Details button 1130 of the pop-up notification window 1120 allows the user to examine the details of the meal along with related information and make modifications and calculations as necessary. To access the details related to the task 1020 when the notification is not present, the user may touch the task icon on the analog clock (e.g., meal icon 1020 shown in FIG. 10). (As used herein, “touch” refers to operation of a touch-screen display of the mobile device 7 in a manner that actuates the touch screen, for example directly touching the screen with a finger or using an intermediate device such as a capacitive-tip pen. Moreover, the user input selecting the icon can be other than a touch screen operation, such as using a tab key to cycle through the selectable options shown in the mobile device display until the icon is highlighted and then selecting the highlighted icon.).

FIG. 12 shows the details that appear if details button 1130 is pressed (or, equivalently, if the icon 1020 is pressed). In box 1, the user enters a current blood sugar level. Alternatively, if communication is enabled between the app 7a and a blood glucose monitor (glucometer), the value may automatically update once the measurement is completed and wirelessly communicated to the app 7a. For comparison, a previous finger stick blood sugar (FSBS) is also displayed along with a timestamp (appearing just below the “FSBS:” header, text “10 pm FSBS: 145”, where “FSBS” represents “finger stick blood sugar”). If there is any confusion regarding the glucose monitor, the user may obtain additional information by pressing right-arrow button 1220 in box 1. This will display a screen as shown in FIG. 13, which shows images of various glucometers. If the user then selects his/her glucose meter by tapping the appropriate image in the display of FIG. 13, then additional information, such as instructions on how to use the meter, are presented as shown in FIG. 14.

Returning to FIG. 12, the user may also enter the carbs (i.e. carbohydrate intake) they plan to eat (e.g., for breakfast). If the number of carbs is known, the user may enter the number directly; if not known, the user may press the right-arrow button 1230 to display additional meal information as shown in FIG. 15. This figure illustrates a screen where the user may select a meal from a list of meals that the user commonly consumes and/or has pre-saved. From this screen, the user may also enter a new meal or may make modifications to one of the existing meals. If the patient eats out frequently, the user may include meals from the restaurants they most frequently patronize; FIGS. 16 and 17 illustrate examples of this.

Once a carb value is entered, a required amount of insulin is calculated and displayed as illustrated in FIG. 18, which shows the same display as FIG. 12 after the FSBS and Carbs values have been entered and the insulin calculated and displayed as “20 units”. The bottommost portion of the display shown in FIG. 18 also shows a diagrammatic patient representation 1240 with the last three insulin injection sites indicated, so as to help the patient rotate injections in accordance with best practice. By selecting the right-arrow located to the right of the diagrammatic patient representation 1240, an enlarged version 1240 L of the patient representation 1240 is brought up, as shown in FIG. 19, via which insulin injection location information for the latest injection can be added by touching the appropriate location on diagram 1240L.

In the embodiment of FIGS. 18 and 19, the insulin is administered via a discrete injection. With brief reference back to FIG. 1, another approach for administering insulin is via an insulin pump 1300, which is a device that delivers a metered insulin dose over time.

With reference to FIGS. 20-23, an embodiment is described in which an insulin pump 1300 is used to administer the insulin. Optionally, the required insulin order may be sent directly to the insulin pump 1300 from the app 7a, as depicted in FIGS. 20-23. As seen in FIG. 20, the diagrammatic patient representation 1240 of FIG. 18 is replaced with a selection button 1250 labeled “Send Insulin Order to Pump”. FIG. 21 shows the display after selection of the button 1250 (where the insulin pump is identified as “OmniPod1”). As this is a therapeutic operation that delivers a drug (insulin) to the patient, the user must perform a confirmation operation as shown in FIG. 21. As shown in FIG. 22, upon confirmation the insulin order is communicated from the device 7 to the insulin pump 1300, and a confirmation message 1260 of the message transmission is displayed on the display of app 7a. FIG. 23 then shows a pop-up message window that is suitably displayed when the insulin pump 1300 sends a message back to the device 7 informing the app 7a that the insulin order has actually been received and executed by the insulin pump 1300.

It will be appreciated that the patient data acquired by, or generated at, the patient app 7a by the various operations described with reference to FIGS. 12-23 are optionally communicated to the trusted third party app(s) 6a running on mobile device(s) 6 possessed by a trusted third party or parties. The app 6a may be variously configured to display this information. In one approach, the read-only app 6a employs the same basic user interface engine as the active app 7a, including the analog clock interface and so forth, but data entry operations are disabled and instead data is “entered” via communications from the active device 7a. In such embodiments, the reminder features, such as the pop-up reminder window 1120 presented in FIG. 11, also appear on the display of the read-only app 6a. In another approach, the read-only app 6a uses a different interface engine, for example presenting a list of self-care tasks with times, indications of whether the task was performed (for past events), and any task data.

Optionally, the app 7a provides statistical, diagnostic, or other data, such as in the following illustrative examples.

FIG. 24 illustrates a view where measured glucose values are displayed graphically in relation to carb intake, exercise, medications, and so forth. The numbers enclosed in circles (from left to right: 44, 15, 37, 48) show values for carbohydrate intake events (e.g. discrete meals). This view demonstrates to a user how well the blood glucose is being managed (e.g., whether the user kept blood glucose levels within a range recommended by a doctor).

As previously noted, HIPAA or other patient privacy considerations may require that the patient consent to any medical data sharing. FIGS. 25 and 26 illustrate an example of a user interface for this purpose, where FIG. 25 shows selection of the “Contacts” window from the main menu, and FIG. 26 shows the contacts window that is brought up by this operation. In one aspect, contacts may view elements of the user's medical information. This is a different modality of medical data sharing than that provided by the read-only app 6a for example, here the medical data may be shared by periodic emails, or by sending the data to a patient database stored at the main server 100 (see FIG. 1), or so forth. The lower list (titled “Share MyHealthView with”) identifies the trusted third persons who can monitor the patient via an instance of the read-only app 6a.

The apparatuses and techniques disclosed herein assist patients at home to manage their own health and administer their own medications safely and easily in order to manage a chronic disease. Diabetes is only one example of a condition that the techniques described herein are useful for treating. The techniques described herein could readily be configured for many chronic conditions such as chronic obstructive pulmonary disease (COPD), attention deficit hyperactivity disorder (ADHD), congestive heart failure (CHF), asthma, bronchitis, depression, bipolar disorder, cancer, cystic fibrosis, autism, etc.

One aspect described herein is to coordinate diverse information needed for home care into a convenient, easy to use interface. Multiple tasks and activities are combined into one convenient, intuitive, easy to use and understand user interface suitably embodied by a mobile device app 7a executable on a cellular telephone or other mobile device 7. The mobile app 7a may be coordinated with a patient leaving the hospital who is prescribed medications or other self-care treatment tasks.

It will be further appreciated that the patient self-care assist techniques disclosed herein may also be embodied by a non-transitory storage medium storing instructions readable and executable by an electronic data processing device to perform the disclosed techniques. Such a non-transitory storage medium may comprise a solid state memory, flash memory, secure digital (SD) card, or other electronic memory of the mobile device 7 (or alternatively a magnetic or optical storage medium if employed in the mobile device 7). Additionally or alternatively, the non-transitory storage medium may comprise a cloud-based storage medium such as a RAID disk array or other network server storage of a mobile OS application store.

The illustrative user interface of the app 7 is to be understood as a non-limiting example. Other user interfaces are also contemplated, such as a grid-style schedule of the type sometimes used in calendar programs, in which time intervals (e.g. hourly intervals) are represented by rows of the grid and the task icons are placed into cells of the grid. As another contemplated variant, the analog clock face display can be retained, but set up as a 24-hour clock face rather than the illustrative conventional 12-hour clock face. As yet another contemplated variant, the grid-style schedule can be used but displaying only a (optionally user selectable) sliding time window running (for example) from one hour in the past to three hours into the future. Such a sliding window enables the user to see in relatively large font or size and focus upon those events of the recent past (up to one hour past in this example) and near future (up to three hours in this example).

The use of images 4, 5, 11 of actual medications and medical devices is advantageous in order to reduce the likelihood of patient error. However, in alternative embodiments it is contemplated to omit such images and display the requisite medication and medical device information in another form, e.g. using textual names of medications and brand names/model numbers of medical devices or so forth. The illustrative connection to the doctor's office 2 and pharmacy 3 is also optional in their place, the user can manually input the names of the prescribed medications and medical devices along with dosages and other information.

If a wireless connection to the pharmacy 3 is provided, another contemplated aspect of the disease management support system is to provide a mechanism to track and refill medicines. In a suitable approach, when the pharmacy 3 fills a prescription for the patient it transmits the number of pills (or inhaler doses, or other dose units) received by the patient to the mobile device 7, and more particularly to the app 7a, which stores this count. Thereafter, each time the patient indicates he or she has self-administered a dose of the medication this count is decremented. At an appropriate time (e.g. when there is one week's worth of doses left) the app 7a automatically sends a refill request to the pharmacy 3. If no refills are available, a request for a new prescription can similarly be wirelessly sent from the app 7a to the doctor's office 2.

Of course, modifications and alterations will occur to others upon reading and understanding the preceding description. It is intended that the invention be construed as including all such modifications and alterations insofar as they come within the scope of the appended claims or the equivalents thereof.

1. A chronic disease management support system comprising:
a mobile device comprising a microprocessor executing an application program to perform a chronic disease management support method including the operations of:
maintaining a disease management schedule of self-care tasks including at least a medication schedule;
displaying a time interval of the disease management schedule including icons representing self-care tasks to be performed during the time interval;
generating an alert when the time for a self-care task arrives;
recording at the mobile device whether each self-care task is performed on schedule; and
in response to user selection of an icon representing a self-care task via a user interface of the mobile device, displaying information pertaining to the self-care task represented by the icon.

2. The chronic disease management support system of claim 1 wherein the mobile device is one of a cellular telephone, a tablet computer, and a personal data assistant (PDA) mobile device.

3. The chronic disease management support system of claim 1 wherein the displaying includes:
displaying an analog clock face representing a twelve hour time interval which indicates a current time, and superimposing icons representing self-care tasks to be performed during the twelve-hour time interval on the analog clock face at locations on the analog clock face corresponding to the scheduled times for the self-care tasks.

4. The chronic disease management support system of claim 1 further comprising:
a monitoring mobile device comprising a microprocessor executing a read-only application program that wirelessly communicates with the mobile device to receive, store, and display at the monitoring mobile device information obtained by the application program executing on the mobile device.

5. The chronic disease management support system of claim 1 wherein the chronic disease management support method includes the further operation of:
wirelessly receiving, at the mobile device, information on at least one medication dose administered by, or medical device used in, performing at least one self-care task of the disease management schedule;
wherein the received information includes an image of the medication dose or medical device; and
wherein the operation of displaying information pertaining to a self-care task in response to user selection of an icon representing the self-care task includes displaying the image of the medication dose or medical device in response to selection of an icon representing the at least one self-care task that administers or uses the medication dose or medical device.

6. The chronic disease management support system of claim 1 wherein the chronic disease management support method comprises a diabetes management support method for which the self-care tasks further include insulin administration tasks, and the diabetes management support method further includes the operations of:
receiving, at the mobile device, blood sugar level and carbohydrate intake information;
using the microprocessor of the mobile device, computing an insulin dose based on the received blood sugar level and carbohydrate intake information; and
displaying the computed insulin dose on a display of the mobile device.

7. The chronic disease management support system of claim 7, wherein the receiving of the carbohydrate intake information includes:
storing, at the mobile device, a list of meals along with carbohydrate information for each meal;
displaying the list of meals on the display of the mobile device; and
receiving, via a user interface of the mobile device, a selection of a meal from the displayed list of meals, whereby the received carbohydrate intake information is the carbohydrate information for the selected meal.

8. The chronic disease management support system of claim 6, wherein the diabetes management support method further includes the operations of:
displaying a diagrammatic patient representation on a display of the mobile device; and
receiving an indication of an insulin injection location based on a user touch of the insulin injection location on the diagrammatic patient representation;
wherein the recording of whether each self-care task is performed on schedule includes recording the received indication of the insulin injection location.

9. The chronic disease management support system of claim 6 further comprising:
an insulin pump;
wherein the diabetes management support method further includes the operations of wirelessly transmitting the computed insulin dose to the insulin pump, and wirelessly receiving confirmation of receipt of the transmitted insulin dose from the insulin pump.

10. A non-transitory storage medium storing instructions readable and executable by a mobile device that includes a microprocessor to perform a method including:
receiving, at the mobile device, prescription information of a patient including a medication type and a medication dosage;
storing the prescription information in a memory of the mobile device; and
based on the stored prescription information, displaying a scheduled time for a dose of medication on a display of the mobile device.

11. The non-transitory storage medium according to claim 10, wherein the displaying includes:
displaying an analog clock face which indicates a current time, and an icon representing the scheduled time for the dose of medication superimposed on the analog clock face at a location on the analog clock face corresponding to the scheduled time.

12. The non-transitory storage medium according to claim 10, wherein the received prescription information further includes an image of the medication and the method further includes:
in response to a user input to the mobile device indicating a request for the prescription information, displaying the image of the medication.

13. The non-transitory storage medium according to claim 12, wherein displaying the image of the medication includes:
if the medication dosage is less than a complete pill amount, displaying an image of a broken pill; and
if the medication dosage is not less than a complete pill amount, displaying an image of a complete pill.

14. The non-transitory storage medium according to claim 12, wherein the user input to the mobile device vindicating a request for the prescription information is a user selection of the icon representing the scheduled time for the dose of medication superimposed on the analog clock face.

15. The non-transitory storage medium according to claim 10, wherein the receiving includes:
receiving the prescription information via wireless communication.

16. The non-transitory storage medium according to claim 10, wherein the method is a diabetes management support method further including:
displaying a scheduled time for an insulin administration on the display of the mobile device;
receiving, at the mobile device, blood sugar level and carbohydrate intake information for the patient;
using a microprocessor of the mobile device, computing an insulin dose based on the received blood sugar level and carbohydrate intake information for the patient;
displaying the computed insulin dose on a display of the mobile device; and
receiving and storing at the mobile device an indication that the scheduled insulin administration has been performed.

17. The non-transitory storage medium according to claim 16, wherein the receiving of the carbohydrate intake information for the patient includes:
storing, at the mobile device, a list of meals along with carbohydrate information for each meal;
displaying the list of meals on the display of the mobile device; and
receiving, via a user interface of the mobile device, a selection of a meal from the displayed list of meals, whereby the received carbohydrate intake information is the carbohydrate information for the selected meal.

18. The non-transitory storage medium according to claim 16, wherein the receiving at the mobile device of an indication that the scheduled insulin administration has been performed includes:
displaying a diagrammatic patient representation on a display of the mobile device; and
receiving an indication of an insulin injection location based on a user touch of the insulin injection location on the diagrammatic patient representation.

19. A chronic disease management support method comprising:
displaying a twelve-hour time interval of a disease management schedule of self-care tasks on a mobile device by displaying an analog clock face representing the twelve hour time interval which analog clock face displays a current time, and superimposing icons representing self-care tasks to be performed during the twelve-hour time interval on the analog clock face at locations on the analog clock face corresponding to the scheduled times for the self-care tasks;
generating an alert when the time for a self-care task arrives;
recording at the mobile device whether each self-care task is performed on schedule; and
in response to user selection of an icon representing a self-care task via a user interface of the mobile device, displaying information pertaining to the self-care task represented by the icon.

20. The chronic disease management support method of claim 19 further comprising:
replicating the display of the twelve hour time interval on a monitoring mobile device including the display of the analog clock face and the superimposed icons;
communicating the record of whether each self-care task is performed on schedule from the mobile device to the monitoring mobile device; and
displaying at the monitoring mobile device at least a portion of the record of whether each self-care task is performed on schedule.
